(19) United States
(12) Patent Application Publication
(10) Pub. No.: US 2017/0363566 A1
(43) Pub. Date: Dec. 21, 2017

(54) Method of Determining Parameters of a Test Fluid

(71) Applicant: INSIDE BIOMETRICS LIMITED, Dingwall (GB)

(72) Inventor: Stephen Blythe, Dingwall (GB)

(21) Appl. No.: 15/534,837

(22) PCT Filed: Dec. 10, 2015
(86) PCT No.: PCT/GB2015/053805

§ 371 Date: Jun. 9, 2017

(30) Foreign Application Priority Data
Dec. 11, 2014 (GB) 1422028.9

(51) Int. Cl. G01N 27/327; G01N 33/49

(52) U.S. Cl. CPC G01N 27/3274; G01N 33/49

(57) ABSTRACT

Determining first and second parameters of a fluid sample includes obtaining a first data set including data from output signals as a function of pluralities of the first and second parameters. The method includes applying an autocorrelation function to the output signals set so as to obtain a second data set including data from a plurality of autocorrelation signals as a function of the pluralities of the first and second parameters. The method includes generating a test output signal at a device by reacting the device with the fluid sample, applying the autocorrelation function to the test output signal so as to obtain a test autocorrelation signal, identifying in the first and second data sets an intersection of data from the test output signal with corresponding data from the test autocorrelation signal, and determining the first and second parameters of the sample based on the intersection.

### FIELD OF TECHNOLOGY

The present disclosure relates to a method of determining parameters of a test fluid. In specific embodiments the test fluid is blood and the parameters are a concentration of an analyte and the haematocrit.

### BACKGROUND

In the field of diagnostic and monitoring devices as used in the medical device industry, especially those used for analysing blood or other bodily fluid samples, it is often required for users to monitor biometrics such as the levels of certain chemicals, substances, or analytes present in their bloodstream. For instance, diabetics in particular must regularly monitor the concentrations of glucose in their blood in order to determine if they are in need of insulin. In order to respond effectively to an individual's needs to monitor blood sugar levels, diagnostic and monitoring devices and kits have been developed over the years to allow an individual to autonomously determine the concentration of glucose in their bloodstream, in order to better anticipate the onset of hyperglycaemia or hypoglycaemia and take preventative action as necessary. The existence of such diagnostic and monitoring devices places less strain on the healthcare system at large, as patients are able to administer insulin in their own home and without having to do so in the presence of a medical professional.

Typically the patient will, using a lancing device, perform a finger stick to extract a small drop of blood from a finger or alternative site. An electrochemical test device, which is often a test strip, is then inserted into a diagnostic/monitoring meter, and the blood sample is applied on the test strip. Through capillary action, the blood sample flows across a measurement chamber of the device and into contact with one or more electrodes or similar conductive elements coated with sensing chemistry for interacting with a particular analyte or other specific chemical (for example glucose) in the blood sample. The magnitude of the reaction is dependent on the concentration of the analyte in the blood sample. The meter may detect the current generated by the reaction of the reagent with the analyte, and the result can be displayed to the user.

Obtaining an accurate analyte concentration measurement is of high importance, as the reading output by the meter forms the basis on which the user may or may not take action such as the administration of insulin. However, the volume of red blood cells (known as the haematocrit) can vary considerably from one blood sample to the next, and therefore is a significant source of inaccuracy to the analyte concentration measurement, and is generally unknown at the time of measurement. Although meters are typically calibrated in order to relatively accurately measure analyte concentration for blood within a certain range of haematocrit, in certain instances the haematocrit may be outside this range. Therefore, the current measurement may be susceptible to systematic inaccuracy the further the blood haematocrit is from the range of values at which the system was calibrated.

One approach of improving the accuracy of the currents measurements obtained is to reduce the haematocrit range within which the meter is calibrated. This however often leads to an undesirably narrow range of haematocrit within which the meter is calibrated to operate, and in general it is desirable to have a meter with relatively high accuracy over a wide range of haematocrit.

There is therefore a need in the art to relatively accurately measure both haematocrit and analyte concentration in a sample of blood, without sacrificing the dynamic range over which the system is calibrated. More generally, when testing a sample of test fluid, there is a need to more accurately measure different parameters of the test sample, where the parameters are unknown and mutually affect readings taken of each other.

### SUMMARY OF THE DISCLOSURE

In a first aspect of the disclosed embodiments, there is provided a method of determining first and second parameters of a test sample of a test fluid. The method comprises obtaining a first data set. The first data set comprises data from a plurality of output signals as a function of pluralities of the first and second parameters. Each output signal is representative of an output signal generated at a test device reacting with a corresponding sample of the test fluid. The method further comprises applying an autocorrelation function to the plurality of output signals set so as to obtain a second data set. The second data set comprises data from a plurality of autocorrelation signals as a function of the pluralities of the first and second parameters. The method further comprises generating a test output signal at a test device by reacting the test device with the test sample of the test fluid. The method further comprises applying the autocorrelation function to the test output signal so as to obtain a test autocorrelation signal. The method further comprises identifying in the first and second data sets an intersection of data from the test output signal with corresponding data from the test autocorrelation signal. The method further comprises determining the first and second parameters of the test sample based on the intersection.

The first data set may be obtained through various different means. For example, the first data set may be obtained by reacting each of the plurality of samples of the test fluid with the test device. Thus, multiple samples of the test fluid may be obtained, and each sample may be individually reacted with a test device in order to produce a number of output signals. In such a case each sample would have a known first parameter and a known second parameter. Each output signal would therefore be representative of a real response signal (or response transient as occasionally referred to in the art) generated at the test device. Alternatively, the first data set may be obtained by modelling reactions of a test device with a plurality of samples of the test fluid. The resultant output signals would therefore be virtual response signals, and the data from which may be used to form the first data set. Thus, each output signal of the first data set does not have to be a real output signal generated under real test conditions, but may instead be merely indicative or representative of such a test.

The first data set may be pre-stored in the meter, for example during manufacturing of the meter. In addition, the results of any test carried out by the test device may be stored for future use, and data from any such tests may be added to the first data set so as to further refine and supplement the first data set.

The data comprised in the first data set may be any number of readings taken from each of the output signals. For example, in the case of the output signal being a current/time transient representative of a reaction between an electrochemical test device and the test fluid, the data comprised in the first data set may be a current reading taken from each output signal or transient. The current reading may be a current reading taken at a predetermined time following acquisition of (or the beginning of) the output signal (known as an end current).

Each autocorrelation signal may represent a degree or extent of correlation between the output signal, and the first and second parameters, as a function of the time lag between them. Autocorrelation may be defined as a measure of correlation between values on the same time series at each of fixed number of lags (1, 2, 3 . . . ). The data comprised in the second data set may be autocorrelation coefficients measured at a specific lag, for each autocorrelation signal.

The test output signal may be any measurable or quantifiable response generated at the test device. In particular, the test output signal may be a current/time transient generated at the test device with the test sample. Like the autocorrelation signal, the test autocorrelation signal may represent a degree or extent of correlation between the test output signal, and the first and second parameters of the test sample, as a function of the time lag between them.

The identification step and the determination of the first and second parameters of the test sample may comprise estimating or determining for which specific values of the first and second parameters the data from the test output signal and the data from the autocorrelation signal meet. One method of achieving this is to represent the data comprised in the first and second data sets as contours or a surface, and to identify an intersection of specific contours and/or surfaces.

Thus, according to the above method, the test transient is run through an autocorrelation function, with the resultant autocorrelation data being compared to autocorrelation data obtained for multiple transients generated from samples having known first and second parameters. Advantageously, relatively accurate and simultaneous estimates of the (unknown) first and second parameters of the test sample may then be obtained.

The estimate may be used to reduce any perceived sensitivity of the test signal to the first and second parameters. In the case of the test fluid being blood, an estimate of haematocrit may be obtained simultaneously to an estimate of the analyte concentration. Knowledge of the haematocrit can be used as the basis for more accurate analyte readings, which is especially important when informing the user of the meter, if for example the reading is to be used as the basis for determining whether or not insulin should be administered. In addition, the haematocrit estimate may also be used as an input for other measurements carried out on a test device. For example, the haematocrit estimate may be used to assist in providing more accurate measurements relating to other analytes present in the fluid sample, for example as would react with other electrodes. The haematocrit value may be reported to the user, for example on a display of the meter or apparatus. Haematocrit is a useful biometric parameter which can be monitored as required, providing useful insight to the user.

Each output signal may comprise a plurality of output values as a function of time, and the first data set may comprise a plurality of output values at a specific time as a function of the pluralities of the first and second parameters.

For example, as explained above, the output signal may comprise a number of current readings (or readings of another electrical characteristic) as a function of time. End current is the current reading or measurement taken at a predetermined time point starting from when a current is first generated at the test device, and the first data set may comprise a plurality of end current readings as a function of the pluralities of the first and second parameters. Thus, in the case where the first and second parameters are analyte concentration and haematocrit, the first data set may comprise information relating end current to analyte concentration and haematocrit.

Each autocorrelation signal may comprise a plurality of autocorrelation values as a function of lag, and the second data set may comprise a plurality of autocorrelation values at a specific lag as a function of the pluralities of the first and second parameters.

For example, each autocorrelation signal may comprise a number of autocorrelation values as a function of lag. Lag may be the distance, in terms of the number of intervening measurements, between two measurements on the transient. For example, for a lag of 3, the two measurements may be the 1^{st} and 4^{th} measurements, the 2^{nd} and 5^{th} measurement, the 3^{rd} and 6^{th} measurements, etc. The first data set may therefore comprise autocorrelation values for a specific lag, as a function of the pluralities of the first and second parameters. Thus, when the first and second parameters are analyte concentration and haematocrit, the second data set may comprise information relating autocorrelation values (for a specific lag) to analyte concentration and haematocrit.

Identifying the intersection may comprise identifying an intersection of the plurality of output values at the specific time with the plurality of autocorrelation values at the specific lag. The first and second data sets may comprise a plurality of contours as a function of the pluralities of the first and second parameters, and identifying the intersection may comprise identifying an intersection of a contour of the first data set with a contour of the second data set. Identifying the intersection may also comprise using numerical analysis to solve equations representing the data from the first and second data sets.

The specific time (or test time) may for example be approximately 5 seconds from when the output signal is first generated at the test device reacting with the corresponding sample of the test fluid. The test time is generally selected as a function of the characteristics of the reagent formulation (coating one or more electrodes of the test device) and performance requirements. For example, a more complex reagent formulation giving a thicker and denser reagent pad matrix will have slower diffusion/kinetic properties and will generally require a longer test time. A balance should be struck between choosing a test time that is not too long so as to inconvenience the user whilst not being too short so as to preclude accurate current readings.

The specific lag may be selected based on a dissimilarity between the plurality of output values at the specific time and the plurality of autocorrelation values at the specific lag. Thus, the specific lag may be selected based on the fact that the second data set will comprise data having a relative dissimilarity to the data of the first data set. Because the first and second data sets each represent respective data as a function of common first and second parameters, variation of data (for example end current) in the first data set may easily be compared to variation of data (for example an autocorrelation value) in the second data set. The greater the dissimilarity between the two, the more information can be extracted to help in the estimate of the first and second parameters.

The dissimilarity may comprise a dissimilarity between a variation of the plurality of output values at the specific time as a function of the pluralities of the first and second parameters, and a variation of the plurality of autocorrelation values at the specific lag as a function of the pluralities of the first and second parameters. For instance, consider the example of the first parameter being analyte concentration, the second parameter being haematocrit, and the first data set relating end current to analyte concentration and haematocrit. If in the first data set the end current decreases as a function of haematocrit, then, when obtaining the second data set, a lag should be selected such that the autocorrelation values or coefficients increase as a function of haematocrit. This may allow a more accurate determination of the first and second parameters, by maximising the dissimilarity between the first and second data sets.

The test device may be an electrochemical test device. For example, the electrochemical test device may be an electrochemical test strip as used in electrochemical assays.

As already mentioned, the first parameter may be a concentration of an analyte in the test sample, and the second parameter may be haematocrit (particularly if the test fluid is whole blood). Other second parameters could be the concentration of other interfering components, such as electrochemical interferents (both exogenous such as drugs/their metabolites, and endogenous such as uric acid). The presence of such interferents in the test fluid can give rise to background current levels, reducing the signal:noise ratio of the output and reducing sensitivity and accuracy.

The analyte may be any of glucose, ketone, lactate, glycerol and cholesterol. Other analytes fall within the scope of the disclosure, such as any analyte the presence of which may be tested for in a fluid sample.

The test fluid may be any of blood, plasma, urine, saliva, lacrimal fluid, sweat, interstitial fluid; and breath condensate. In some of these test fluids the second parameter may be for example the volume of albumin, proteins, lipids, cholesterol, triglycerides, etc.

In a second aspect of the disclosed embodiments, there is provided an apparatus, such as a meter, for reading test devices. The apparatus comprises one or more memories storing a first data set comprising data from a plurality of output signals as a function of pluralities of first and second parameters. Each output signal is representative of an output signal generated at a test device reacting with a corresponding sample of a test fluid. The one or more memories also store a second data set comprising data from a plurality of autocorrelation signals as a function of the pluralities of the first and second parameters. The apparatus further comprises means for reading a test output signal generated at a test device by reacting the test device with a test sample of the test fluid. The apparatus further comprises one or more processors arranged to apply an autocorrelation function to the test output signal so as to obtain a test autocorrelation signal. The one or more processors are further configured to identify in the first and second data sets an intersection of data from the test output signal with corresponding data from the test autocorrelation signal. The one or more processors are further configured to determine the first and second parameters of the test sample based on the intersection.

In the case of a meter, the apparatus may further comprise receiving means for receiving the test device and generating the test output signal at the test device. In the case of the test device being a test strip, the receiving means may be further arranged to apply a potential difference across two or more electrodes of the test strip, as is known in the art.

Any feature of the first aspect (relating to the method of determining the first and second parameters) may be used with the second aspect (relating to the apparatus). The methods and apparatus described above may also be used with any suitable electrochemical test device, such as a test strip or a patch. In the case of a patch, the apparatus may be configured so as to wirelessly collect transient data generated at the patch, for example by bringing the apparatus into close proximity with the patch.

In addition, the electrochemical test device may, for example, be suitable for testing for multiple analytes. When a multi-analyte test device is available, the disclosed methods for determining parameters of a test fluid sample may be used to configure the device to determine parameters of multiple analytes in the test fluid sample. The disclosed methods of determining a concentration of an analyte in a test fluid sample, where the analyte concentration is unknown, may be extended to determine concentrations of multiple analytes in the test fluid sample.

### BRIEF DESCRIPTION OF THE DRAWINGS

Specific embodiments will now be described in connection with the accompanying drawings, of which:

FIG. 1 is a schematic representation of a meter arranged to read an electrochemical test strip, in accordance with an embodiment;

FIG. 2 shows a method of determining parameters of a sample of a test fluid, in accordance with an embodiment;

FIG. 3 shows a first data set showing end current as a function of plasma glucose and haematocrit;

FIG. 4 is a plot of an autocorrelation signal as a function of lag k;

FIG. 5 is a plot of a current transient as a function of time;

FIG. 6 shows a second data set showing autocorrelation values for a lag of 25 as a function of plasma glucose and haematocrit;

FIG. 7 is a plot of the data of FIG. 3 combined with the data of FIG. 6;

FIG. 8 shows a surface representation of the first data set of FIG. 3;

FIG. 9 shows a surface representation of the second data set of FIG. 6;

FIG. 10 is a plot showing end current measurements as a function of plasma glucose;

FIG. 11 is a plot of mean percent bias from glucose reference measurement, as a function of haematocrit;

FIG. 12 is a surface representation of mean current as a function of plasma glucose and haematocrit;

FIG. 13 is a plot of an autocorrelation signal as a function of lag k;

FIG. 14 is a plot of autocorrelation values for a lag of 50 as a function of plasma glucose and haematocrit;

FIG. 15 is a plot of end current as a function of plasma glucose and haematocrit; and

FIG. 16 is a plot of mean percent bias from glucose reference measurement, as a function of haematocrit.

### DETAILED DESCRIPTION OF SPECIFIC EMBODIMENTS

The presently disclosed embodiments seek to provide an improved method of determining parameters of a test fluid. Whilst various embodiments are described below, the contemplated embodiments are not limited to these embodiments, and variations of these embodiments may well fall within the scope of the appended claims.

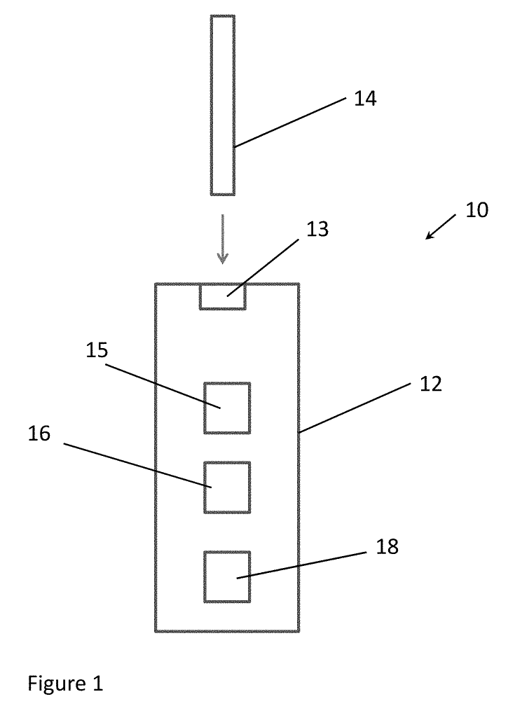

FIG. 1 shows a strip-meter system 10 according to an embodiment. System 10 comprises a meter 12 for reading an electrochemical test strip 14. Electrochemical test strip 14 comprises one or more working electrodes (not shown) and a counter/reference electrode, each of the working electrodes having a reagent coated thereon for reacting with a sample of test fluid to be applied to electrochemical test strip 14. The counter/reference electrode may also have a reagent coated thereon. Meter 12 comprises receiving means 13 for receiving test strip 14 and applying a potential difference between the working electrode(s) and the counter/reference electrode.

Meter 12 further comprises processing circuitry 15 for carrying various functions relating to the operation of meter 12. For example, processing circuitry 15: controls operation of receiving means 13 so to control application of a potential difference between the working electrode(s) and the counter/reference electrode; processes transients generated at test strip 14; controls the display of messages on display 18; etc. Meter 12 further comprises a memory storage 16 and a display 18 for displaying readouts of measurements taken by meter 12.

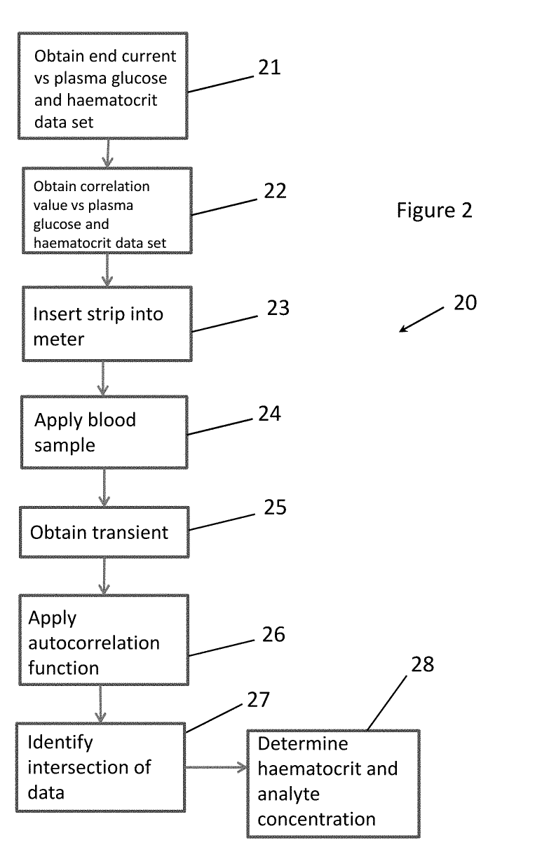

FIG. 2 shows a method of determining parameters of a test fluid, in accordance with an embodiment. It should be noted that FIG. 2 shows an example method, and the order of the steps may be changed (for example the point in time at which the strip is inserted in the meter) without departing from the scope of the disclosed embodiments. The method may also comprise a fewer or greater number of steps.

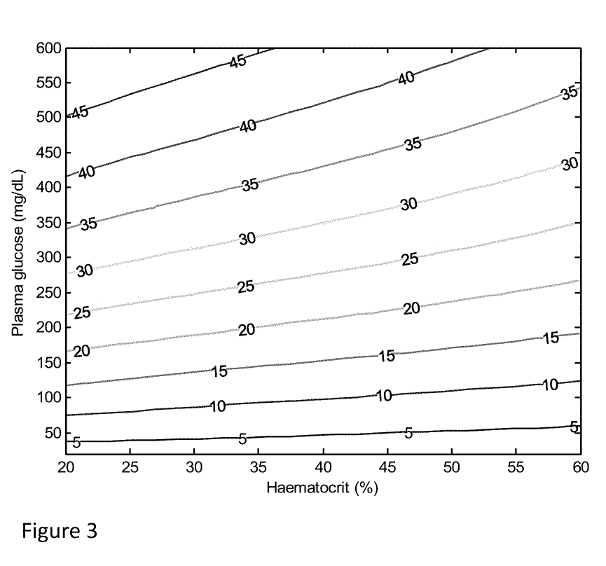

At step 21, a first data set is obtained. The first data set comprises data (e.g. end current) from a plurality of current transients representing current responses generated at an electrochemical test device. The end current is shown as a function of both plasma glucose (e.g. the concentration of glucose within the plasma portion of blood) and haematocrit. In other embodiments, meter 12 could of course be configured to determine the glucose concentration in the whole blood sample (i.e. the glucose content of both the plasma and red blood cells). An example of the first data set is illustrated in FIG. 3, which shows contours of constant end current (μA) as a function of haematocrit and plasma glucose.

It is clear from FIG. 3 that the higher the haematocrit, the lower the end current response for a given plasma glucose, and vice versa. Thus, when a given end current value is obtained, it could have been generated by any combination of glucose and haematocrit along the appropriate contour. For example, a measured end current of 30 μA could mean a glucose concentration between 270 mg/dL and 440 mg/dL. If another quantity or parameter can be measured which has a different sensitivity to haematocrit and plasma glucose than end current, then it is possible to obtain simultaneous estimates of plasma glucose and haematocrit.

The data from FIG. 3 was obtained using a simulated system. A system of reaction-diffusion equations modelling the chemical and physical properties of an electrochemical test strip was developed. Using the model, a set of transients was obtained, with haematocrit and glucose values on a grid over the stated range. An algorithm for determining a concentration of glucose within each test sample was then applied to each current transient. Examples of such algorithms are disclosed in co-pending UK patent application no. 1419799.0, which is incorporated herein by reference.

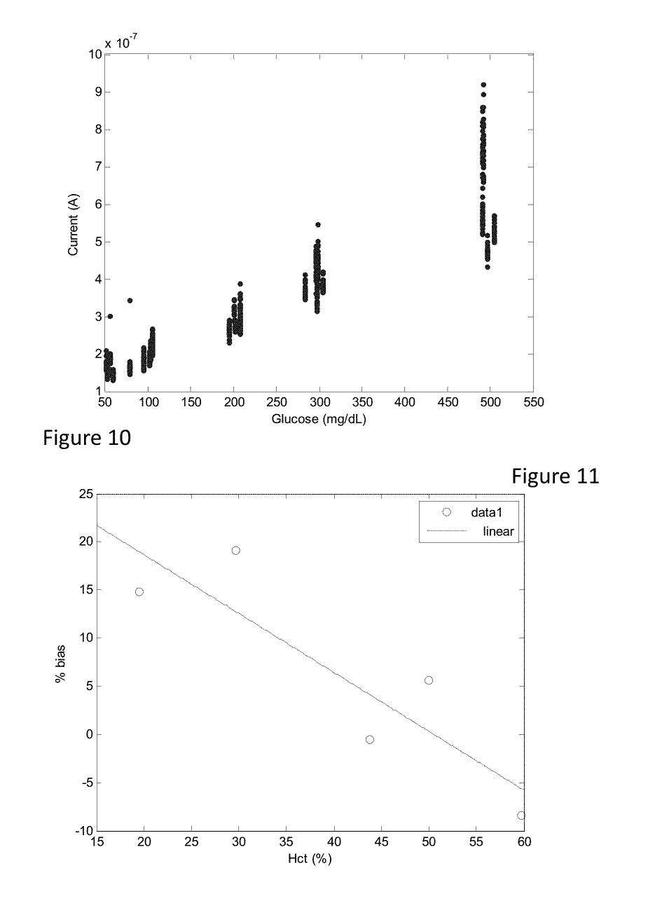

The 5-second current value (known as the end current) from each transient was recorded, in addition to the glucose and haematocrit values used to create the transient. In the present embodiment, the data may be pre-stored in memory 16 of meter 10 for use in the method described in more detail below. However, in other embodiments the data may be ‘real’ data, i.e. data obtained from multiple tests carried out on test samples of various different known haematocrit and plasma glucose levels. Examples of such real data are disclosed below in connection with FIGS. 10-16.

The sample autocorrelation function is a well-known means of measuring the degree of correlation between values in a signal, based on the separation in time between the values. Without loss of generality, assume that the signal has N sequential readings equally spaced in time: x(1), x(2), . . . , x(N). Then, the autocorrelation coefficient r(k) for lag of length k is defined as:

$$r ( k ) = \frac{c ( k )}{c ( 0 )}$$

$$c ( k ) = \frac{1}{N} {\sum }_{t = 1}^{N - k} ( x ( t ) - \overline{x} ) ( x ( t + k ) - \overline{x} ) , \\ k = 0 , 1 , 2 , … , K$$

r(k) is the autocorrelation coefficient for lag k, c(k) is the autocovariance function of the lag k, K is a maximum lag less than N, and x̅ is the mean of the signal readings. A particular c(k) describes the covariance between points in the transient k sample time points apart (lag of k). Not all the c(k) values need to be calculated—only the ones of interest depending on the lag. When scaled by c(0), r(k) is obtained—the autocorrelation coefficient between points k samples apart.

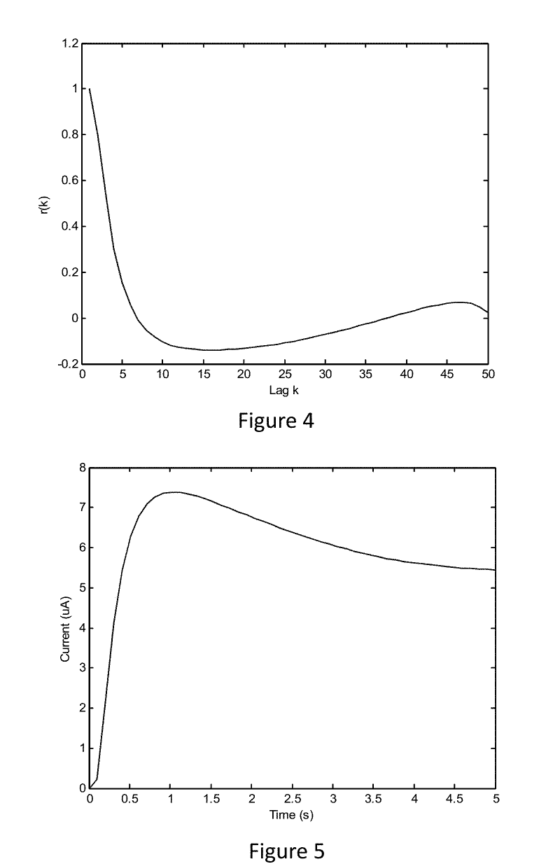

An example autocorrelation plot applied to a transient (real or virtual) is shown in FIG. 4. As can be seen, the r(k) function decreases steadily with lag length and shows some more complicated behaviour towards the end of the plot, rising and then falling again. In the present embodiment, the current/time transient from which the autocorrelation plot of FIG. 4 was obtained is shown in FIG. 5. The transient was obtained for a sample where the plasma glucose was 45 mg/dL and the haematocrit was 30%. 50 measurements of current were taken in a space of 5 seconds.

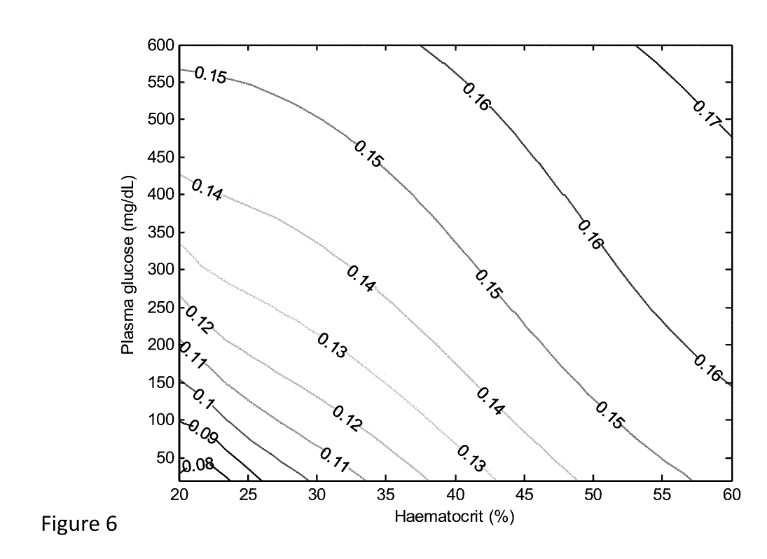

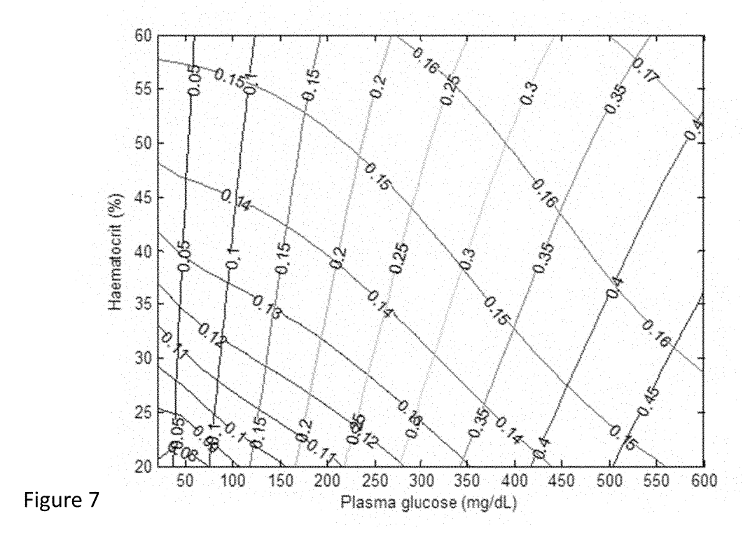

By applying the autocorrelation function to each transient used to obtain the first data set, a second data set is obtained (step 22). An example of the second data set can be seen in FIG. 6 and shows contours of constant autocorrelation coefficient, r, for a specific lag, as a function of haematocrit and plasma glucose. In the present embodiment a specific lag of k=25 was chosen to give a ‘landscape’ (FIG. 6) that is sufficiently distinguished to the 5-second end current contour map of FIG. 3. It is clear that the shape of the autocorrelation coefficient response to haematocrit and plasma glucose is quite different to that of end current as in FIG. 3. In particular, in the end current map of FIG. 3, end current increases as plasma glucose increases, but decreases as haematocrit increases. On the other hand, in the |r(25)| map of FIG. 6, |r(25)| increases as both plasma glucose and haematocrit increase. The intersection of the two data sets can be seen in FIG. 7.

Using the first and second data sets, the method is able to simultaneously determine or at least estimate the plasma glucose and haematocrit for a given test sample of blood. At step 23, electrochemical test strip 14 is inserted into receiving means 13 of meter 12, in a reading position. In the reading position, receiving means 13 is positioned relative to the working electrode(s) of strip 14 so as to be able to apply a potential difference across the working electrode(s) and the counter/reference electrode, as known in the art. Receiving means 13, under control of processor 15, then applies a potential difference across the working electrode(s) and the counter-reference electrode. At step 24, a test sample of blood having unknown plasma glucose and haematocrit is applied to strip 14. As known in the art, a current/time transient is generated as the blood flows into contact with the working electrode(s) and the counter/reference electrode. The glucose in the blood reacts with the reagent on the working electrode(s), and causes a current to flow between the working electrode(s) and the counter/reference electrode. At step 25 the current response is measured by the meter using processor 15. It should be understood that other analytes in the blood may be measured, such as ketones, lactate, glycerol or cholesterol, and that in the present embodiment glucose is merely used as an example.

Once collected, at step 26 the autocorrelation function is applied to the test transient as explained above, thereby obtaining an autocorrelation coefficient r(k). The end current and r surfaces can be approximated by suitable functions. By way of example, the surfaces may be represented in polynomial form as per the below:

$$C = {\sum }_{j , k} {p}_{j , k} {X}^{j} {Y}^{k}$$

$$R = {\sum }_{j , k} {q}_{j , k} {X}^{j} {Y}^{k}$$

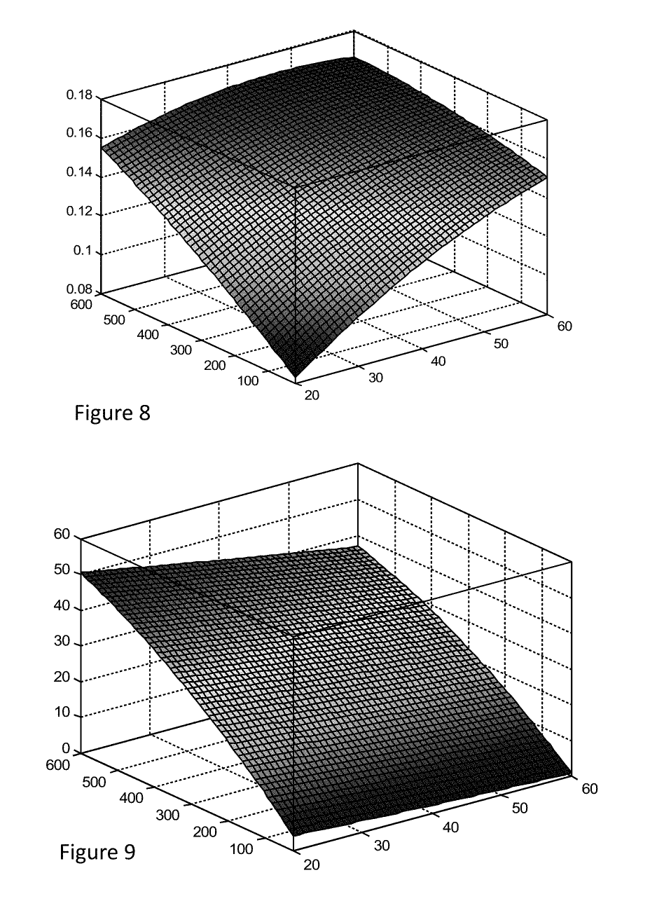

Here, X denotes haematocrit, Y denotes glucose concentration, R=|r(25)| and C denotes end current. X and Y may then be obtained simultaneously using the actual measurements of C and R for the test transient. The C and R contours are characterised as two surfaces in FIGS. 8 and 9, using polynomials with up to second order terms. Numerical procedures for solving simultaneous polynomials are plentiful, and may be implemented in a hand-held device such as meter 12. The general procedure for implementing a Newton-Raphson solver iterates until a solution is known as precisely as it is desired, and may be written compactly in matrix notation as:

Xₙ₊₁=Xₙ−J⁻¹(Xₙ)F(Xₙ)

n is the index of the iteration, X is the vector of the two values to be sought (haematocrit and plasma glucose), F is the vector of equations to be solved, and J⁻¹ is the inverse of the Jacobian matrix of F.

At step 27, processor 15 applies such an iterative method to the C and R surfaces to identify where they intersect, and obtains estimates of the plasma glucose and haematocrit of the blood sample (step 28). Of course, other methods of solving two simultaneous equations with two unknowns may be used. The plasma glucose concentration may be displayed to the user on display 18.

FIGS. 10-16 relate to data obtained from transients generated from real tests on blood samples, as opposed to a simulated system. The figures build on the model-based concept described above by looking at data from physical test strips measuring a particular analyte in blood.

FIG. 10 shows 572 end current values, each taken at the 5-second point from the start of the test, for a range of glucose concentration (50-500 mg/dL) and haematocrit values (20%-60%). In general, a measurement frequency of at least 10 measurements per second, and at least 50 measurements in total, yields appropriate current transients. The measurements should be equally spaced in time. It is clear from FIG. 10 that there is a relatively strong glucose signal as current is substantially proportional to glucose concentration. However, there is also much variation at any given value of glucose concentration.

FIG. 11 shows by way of illustration the mean percent bias from glucose reference measurement for the 500 mg/dL plasma glucose concentration and each of the haematocrit levels (circles), as well as the linear regression line of best fit. This graph also clearly shows a pronounced sensitivity to haematocrit in the signal, albeit with noise.

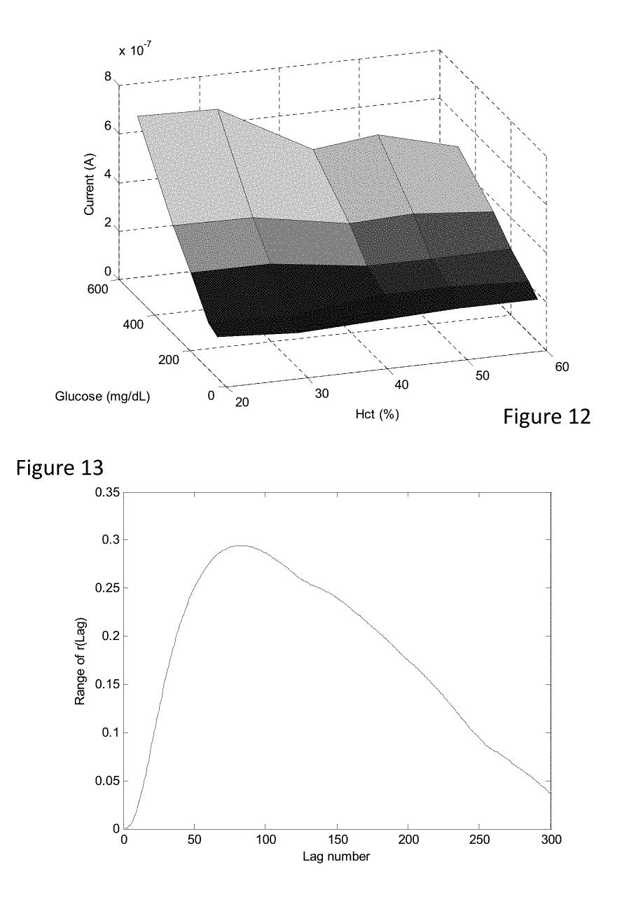

Combining the data from FIG. 10 with FIG. 11 gives FIG. 12, which shows the mean current at the different combinations of glucose and haematocrit. In order to reduce the sensitivity to haematocrit, the autocorrelation function at various lags can be calculated for this data, as described above.

FIG. 13 plots the dynamic range of autocorrelation values obtained at each lag. A possible means of choosing the best lag is to use one from the region of highest range (between 40 to 60 in the example of FIG. 13), and in this case lag k=50 is chosen. This corresponds to a correlation timescale of 0.76 seconds.

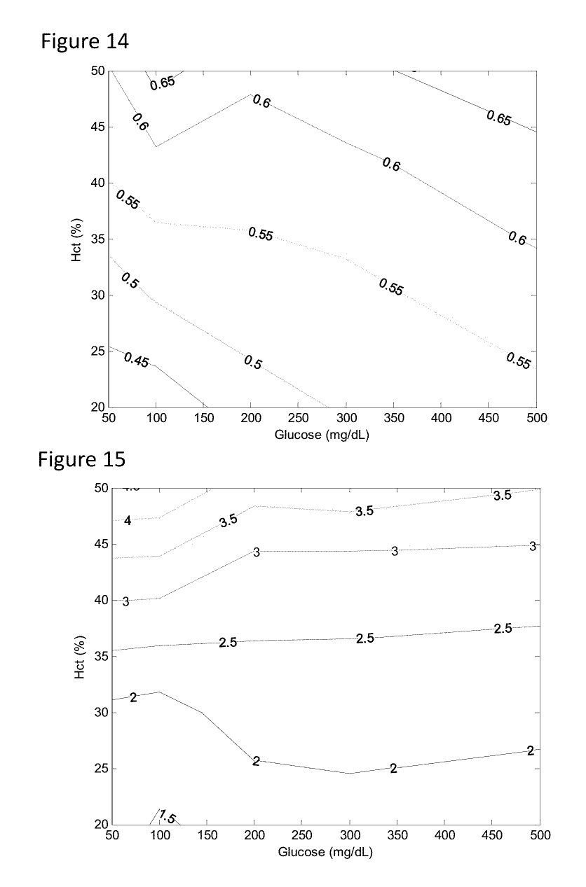

The contour plots of r(50) and 5-second end current (μA) for this data are seen in FIGS. 14 and 15 respectively. Clearly, there exists a portion of the plasma glucose/haematocrit range where a contrary slope to that of the 5-second end current is seen. This dissimilarity between the two contour plots may therefore be exploited in much the same fashion as described above in connection with the simulated system of FIGS. 3-9.

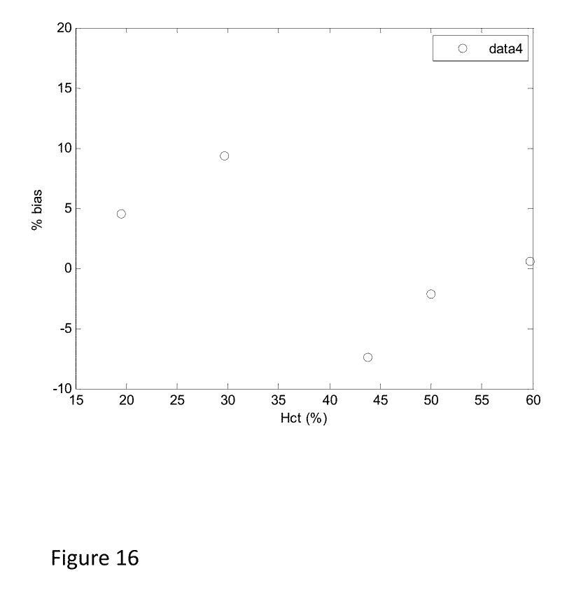

FIG. 16 shows the result of using the two contour plots of FIGS. 14 and 15 to obtain a plot of mean percentage bias against haematocrit, for a 500 mg/dL plasma glucose concentration. In comparison to the plot of uncorrected data of FIG. 11, it is clear that the haematocrit sensitivity has been reduced by two thirds, thereby enhancing the accuracy of the glucose estimation.

Whilst described in connection with specific embodiments, it is to be understood that the contemplated embodiments are not limited to those described, and that alterations, modifications, and variations of these embodiments may be carried out by the skilled person without departing from the scope of the contemplated embodiments. For instance, whilst described primarily in the context of determining parameters of a test fluid, with particular reference to medical devices for measuring glucose in people with diabetes, the contemplated improvements may equally well be used in other fields, for example in health and fitness, food, drink, bio-security applications, environmental sample monitoring, veterinary devices, etc. Thus, instead of using a meter as used in electrochemical assays, it is envisaged that the method could be used with general scientific apparatus suitable for fluid samples.

Furthermore, whilst primarily described in the context of its use with electrochemical test strips, the contemplated improvements may extend to other electrochemical devices, such as wearable devices that actively acquire a fluid sample (such as interstitial fluid) from a user and cause an electrochemical reaction to occur with the sample. Examples of such are continuous (or semi-continuous) glucose monitoring devices used for controlling glucose concentrations (and insulin dosing) by users with diabetes.

1. A method of determining first and second parameters of a test sample of a test fluid, comprising:
obtaining a first data set, the first data set comprising data from a plurality of output signals as a function of pluralities of the first and second parameters, wherein each output signal is representative of an output signal generated at a test device reacting with a corresponding sample of the test fluid;
applying an autocorrelation function to the plurality of output signals set so as to obtain a second data set, the second data set comprising data from a plurality of autocorrelation signals as a function of the pluralities of the first and second parameters;
generating a test output signal at a test device by reacting the test device with the test sample of the test fluid;
applying the autocorrelation function to the test output signal so as to obtain a test autocorrelation signal;
identifying in the first and second data sets an intersection of data from the test output signal with corresponding data from the test autocorrelation signal; and
determining the first and second parameters of the test sample based on the intersection.

2. The method of claim 1, wherein each output signal comprises a plurality of output values as a function of time, and wherein the first data set comprises a plurality of output values at a specific time as a function of the pluralities of the first and second parameters.

3. The method of claim 2, wherein each autocorrelation signal comprises a plurality of autocorrelation values as a function of lag, and wherein the second data set comprises a plurality of autocorrelation values at a specific lag as a function of the pluralities of the first and second parameters.

4. The method of claim 3, wherein identifying the intersection comprises identifying an intersection of the plurality of output values at the specific time with the plurality of autocorrelation values at the specific lag.

5. The method of claim 2, wherein the specific time is approximately 5 seconds from when the output signal is first generated at the test device reacting with the corresponding sample of the test fluid.

6. The method of claim 3, wherein the specific lag is selected based on a dissimilarity between the plurality of output values at the specific time and the plurality of autocorrelation values at the specific lag.

7. The method of claim 6, wherein the dissimilarity comprises a dissimilarity between a variation of the plurality of output values at the specific time as a function of the pluralities of the first and second parameters, and a variation of the plurality of autocorrelation values at the specific lag as a function of the pluralities of the first and second parameters.

8. The method of claim 1, wherein the test device is an electrochemical test device.

9. The method of claim 1, wherein the first parameter is a concentration of an analyte in the test sample.

10. The method of claim 9, wherein the analyte is any one of: glucose, ketone, lactate, glycerol and cholesterol.

11. The method of claim 1, wherein the test fluid is blood and wherein the second parameter is the haematocrit of the test sample.

12. The method of claim 1, wherein the first data set is obtained by modelling the reactions of the test device with the plurality of samples of the test fluid.

13. The method of claim 1, wherein the first data set is obtained by reacting each of the plurality of samples of the test fluid with the test device.

14. The method of claim 1, wherein identifying the intersection comprises using numerical analysis to solve equations representing the data from the first and second data sets.

15. The method of claim 1, wherein the test output signal comprises a current generated at the test device.

16. An apparatus, comprising:
one or more memories storing:
a first data set comprising data from a plurality of output signals as a function of pluralities of first and second parameters, wherein each output signal is representative of an output signal generated at a test device reacting with a corresponding sample of a test fluid; and
a second data set comprising data from a plurality of autocorrelation signals as a function of the pluralities of the first and second parameters;
means for reading a test output signal generated at a test device by reacting the test device with a test sample of the test fluid; and
one or more processors arranged to:
apply an autocorrelation function to the test output signal so as to obtain a test autocorrelation signal;
identify in the first and second data sets an intersection of data from the test output signal with corresponding data from the test autocorrelation signal; and
determine the first and second parameters of the test sample based on the intersection.

17. A computer-readable medium having instructions stored thereon, wherein the instructions are configured when executed to cause a computer to:
obtain a first data set, the first data set comprising data from a plurality of output signals as a function of pluralities of the first and second parameters, wherein each output signal is representative of an output signal generated at a test device reacting with a corresponding sample of the test fluid;
apply an autocorrelation function to the plurality of output signals set so as to obtain a second data set, the second data set comprising data from a plurality of autocorrelation signals as a function of the pluralities of the first and second parameters;
generate a test output signal at a test device by reacting the test device with the test sample of the test fluid;
apply the autocorrelation function to the test output signal so as to obtain a test autocorrelation signal;
identify in the first and second data sets an intersection of data from the test output signal with corresponding data from the test autocorrelation signal; and
determine the first and second parameters of the test sample based on the intersection.

18. (canceled)
